(19) United States
(12) Patent Application Publication
(10) Pub. No.: US 2004/0146912 A1
(43) Pub. Date: Jul. 29, 2004

(54) Detecting a nucleic acid

(76) Inventors: Allan Lipton, Hershey, PA (US); Kim Leitzel, Hummelstown, PA (US); Michael F. Verderame, Hershey, PA (US)

Correspondence Address: Stanley A. Kim, Ph.D., Akerman Senterfitt, Suite 400, 222 Lakeview Avenue, West Palm Beach, FL, 33401-6183, US

(21) Appl. No.: 10/740,091

(22) Filed: Dec. 18, 2003

Related U.S. Application Data
(60) Provisional application No. 60/435,538, filed on Dec. 18, 2002.
(60) Provisional application No. 60/504,851, filed on Sep. 22, 2003.

(51) Int. Cl. C12Q 1/68

(52) U.S. Cl. 435/6

(57) ABSTRACT

Methods and compositions for detecting a nucleic acid correlated with the presence of breast cancer in a human subject are described herein.

### CROSS-REFERENCE TO RELATED APPLICATIONS

The present application claims the priority of U.S. provisional patent application serial No. 60/435,538 filed on Dec. 18, 2002 and U.S. provisional patent application serial No. 60/504,851 filed on Sep. 22, 2003.

### FIELD OF THE INVENTION

The invention relates generally to the fields of molecular biology and clinical diagnostics. More particularly, the invention relates to a method of detecting a mutant nucleic acid contained in a biological sample to aid in the diagnosis or determining the prognosis of a patient having a disease or predisposed to acquiring a disease associated with a mutant nucleic acid.

### BACKGROUND

Although breast cancer remains a major cause of death worldwide, over the last few decades, significant progress has been made in methods for both diagnosing and treating the disease. Much of this progress has been related to the identification of wild-type (WT) and mutant markers associated with breast cancer, e.g., estrogen receptor (ER), BRCA1, BRCA2, CEA, CA 15-3, CA 27.29 and HER-2/neu. Despite these advances, there is currently no serum diagnostic assay for early detection of breast cancer in the general population that is both sensitive enough and sufficiently specific to allow for the screening, diagnosis, and/or staging of breast cancer patients after primary therapy.

### SUMMARY

The invention relates to methods and compositions for detecting a nucleic acid correlated with the presence of or predisposition to acquiring breast cancer in a human subject. In the examples described below, a polymerase chain reaction (PCR)-based method was created to detect mutant ER DNA associated with breast cancer (A to G transition in ER) in serum or plasma from a human subject. The approach employs a nested PCR reaction with a restriction endonuclease digestion between the two PCR rounds that is specific for WT ER DNA. Digestion with a WT DNA-specific enzyme reduces the level of WT template, and thus increases the sensitivity of the assay for mutant DNA. Detection of ER DNA carrying the mutation is accomplished via mutation-specific restriction endonuclease digestion and gel electrophoresis. Femtograms of mutant ER DNA can be detected in the presence of 100-fold excess of WT DNA. The extreme sensitivity of this assay allows it to be used in samples (e.g., blood) that contain only minute amounts of the DNA being screened for.

Accordingly, the invention features a method for detecting a nucleic acid correlated with the presence of or predisposition to acquiring breast cancer in a human subject. The method includes the step of detecting in a bodily fluid of the subject a nucleic acid having an A to G transition at nucleotide 908 in the estrogen receptor gene, wherein the presence of the nucleic acid comprising an A to G transition at nucleotide 908 indicates the subject has or is predisposed to acquiring breast cancer. In preferred variations of the invention, the bodily fluid includes blood or a blood fraction (e.g., plasma, serum).

The step of detecting the nucleic acid can be performed by: (a) subjecting nucleic acids from the bodily fluid to a first polymerase chain reaction resulting in a first population of polymerase chain reaction products including at least a first nucleic acid having an A to G transition at nucleotide 908 in the estrogen receptor gene and a second nucleic acid WT at nucleotide 908 in the estrogen receptor gene, (b) subjecting the first population of polymerase chain reaction products to a first restriction enzyme digest, wherein the enzyme recognizes a restriction site overlapping nucleotide 908 in the second nucleic acid but not in the first nucleic acid, (c) subjecting the first population of polymerase chain reaction products to a second polymerase chain reaction resulting in a second population of polymerase chain reaction products, and (d) subjecting the second population of polymerase chain reaction products to a second restriction enzyme digest, wherein the restriction enzyme recognizes a restriction site overlapping nucleotide 908 in the first nucleic acid but not in the second nucleic acid and is different from the restriction enzyme in the first restriction enzyme digest.

In the foregoing method, the first polymerase chain reaction can include a first oligonucleotide primer and a second oligonucleotide primer, the first oligonucleotide primer having the nucleotide sequence of SEQ ID NO:5, and the second oligonucleotide primer having the nucleotide sequence of SEQ ID NO:6. The second polymerase chain reaction can include a third oligonucleotide primer and a fourth oligonucleotide primer, the third oligonucleotide primer having the nucleotide sequence of SEQ ID NO:7, and the fourth oligonucleotide primer having the nucleotide sequence of SEQ ID NO:8. Nucleic acid fragments of the second restriction enzyme digest are electrophoretically separated to yield an electrophoretic pattern specific to the digestion of the first nucleic acid. The electrophoretic pattern specific to the first nucleic acid indicates the presence of the first nucleic acid in the bodily fluid. The presence of the first nucleic acid in the bodily fluid indicates that the bodily fluid contains a breast cancer.

The invention further features a kit for detecting a nucleic acid correlated with the presence of or predisposition to acquiring breast cancer in a human subject. The kit includes a first pair of oligonucleotides for amplifying a portion of the estrogen receptor gene, a second pair of oligonucleotides for amplifying a nucleic acid within the portion of the estrogen receptor gene amplified by the first pair of oligonucleotides, a first restriction enzyme, a second restriction enzyme, and printed instructions for performing polymerase chain reactions using the first and second pairs of oligonucleotides and restriction digests using the first and second restriction enzymes.

As used herein, “bind,” “binds,” or “interacts with” means that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ moles/liter for that second molecule.

As used herein, a “detectable label” is meant any substance that can be detected either directly or indirectly.

By the phrase “conjugated to” is meant covalently or non-covalently bonded to or otherwise physically associated with.

By the term “gene” is meant a nucleic acid molecule that codes for a particular protein, or in certain cases a functional or structural RNA molecule.

As used herein, a “nucleic acid” or a “nucleic acid molecule” means a chain of two or more nucleotides such as RNA (ribonucleic acid) and DNA (deoxyribonucleic acid).

By the phrase “a subject predisposed to acquiring breast cancer” means that the subject has a statistically higher likelihood of acquiring breast cancer than do other similarly situated (e.g., same sex, same age, same ethnicity) individuals in a given population.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### DETAILED DESCRIPTION

The invention provides a method and kit for detecting a nucleic acid correlated with the presence of or predisposition to acquiring breast cancer in a human subject. The method involves a nested PCR-restriction enzyme digest-based assay to detect mutant ER DNA in a bodily fluid. The methods and kits described herein present promising tools for the screening, diagnosis, staging, and/or routine surveillance of breast cancer patients after primary therapy. Although this technology is particularly useful for detecting the presence of or predisposition to acquiring breast cancer, it may be used for detecting mutant nucleic acids in a bodily fluid in cancer or pre-cancerous conditions in other anatomical locations such as the ovaries and cervix, or for detecting other conditions containing the mutant ER.

The below described preferred embodiments illustrate adaptations of these compositions and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3^{rd} ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Use of restriction enzymes, including digest conditions and restriction sites, is described in the New England Biolabs Catalogue and Technical Reference, New England Biolabs, Inc., Beverly, Mass., 2002-2003. Various techniques using PCR are described, e.g., in Innis et al., PCR Protocols: A Guide to Methods and Applications, Academic Press: San Diego, 1990. PCR-primer pairs can be derived from known sequences by known techniques such as using computer programs intended for that purpose (e.g., Primer, Version 0.5, ©1991, Whitehead Institute for Biomedical Research, Cambridge, Mass.). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers.

### Biological Samples

A preferred embodiment of the invention relates to the detection of a DNA containing an A to G transition at position 908 in the ER gene in a bodily fluid of a subject. The A to G transition at position 908 in the ER gene (position 1 is defined as the beginning of the open reading frame, i.e., the A of the initiator methionine codon) appears in bodily fluids in communication with cancerous cells. Obtaining bodily fluids from a subject is typically much less invasive and traumatizing than obtaining a solid tissue biopsy sample. Thus, samples which are bodily fluids are preferred for use in the invention. Particularly preferred bodily fluids are blood and blood fractions including serum and plasma. In the experiments described below, plasma was used for the detection of mutant ER DNA. Other bodily fluids, however, may also be used for the detection of mutant ER DNA. Such bodily fluids include, but are not limited to: serum, effusions, perfusions, cerebrospinal fluid (CSF), amniotic fluid, breast secretions, nipple aspirates, tumor cell extracts, urine, or any extracellular or cellular fluids. Bodily fluids containing circulating tumor cells may also be used.

A bodily fluid can be obtained from a subject by conventional techniques. For example, blood can be obtained by venipuncture, while plasma and serum can be obtained by fractionating whole blood according to known methods. As another example, CSF can be obtained by lumbar puncture. Although bodily fluids are preferred, the methods and compositions described herein are also useful for determining the presence of the mutation in biopsy specimens, which may aid in diagnosis and treatment decisions. Surgical techniques for obtaining solid tissue samples are well known in the art.

Because the experiments presented herein relate to human subjects, a preferred subject for the methods of the invention is a human being. Particularly preferred are human subjects are those suspected of having or being at increased risk for developing breast cancer. In addition to human beings, the methods of the invention might be extended to a non-human animal subject such as a dog, cat, horse, cow, pig, sheep, goat, chicken, primate, rat, or mouse, in the case that a nucleic acid marker of disease is present in a bodily fluid in the subject.

### Nucleic Acids

A nucleic acid to be detected in a bodily fluid is a nucleic acid (e.g., DNA, RNA) encoding an ER having an A to G transition at position 908. This mutation creates a restriction site overlapping position 908 for restriction enzyme Mnl I that is not present in the WT ER sequence. This mutation also results in the loss of a Mbo II restriction site overlapping position 908 that is present in the WT ER sequence. A portion of the estrogen receptor gene WT at position 908 is the nucleotide sequence of SEQ ID NO:1. WT ER sequence is deposited with Genbank as accession number X03635. A portion of the estrogen receptor gene having the A to G transition at position 908 is the nucleotide sequence of SEQ ID NO:2. The methods and compositions described herein might be modified by known methods to detect in a bodily fluid other nucleic acids associated with disease, e.g., in the case where such nucleic acids are present in only minute quantities (femtogram level) in the bodily fluid.

### Detecting a Nucleic Acid in a Sample

The invention provides a method for detecting a nucleic acid correlated with the presence of or predisposition to acquiring breast cancer in a human subject. The method includes the step of detecting in the bodily fluid of the subject a nucleic acid having an A to G transition at nucleotide 908 in the ER gene. A method of detecting the mutation at position 908 involves several steps. First, nucleic acids from the bodily fluid are subjected to a first PCR resulting in a first population of PCR products including at least a first nucleic acid having an A to G transition at nucleotide 908 in the ER gene and a second nucleic acid WT at nucleotide position 908 in the ER gene. In the first PCR, any standard PCR conditions may be used that result in amplification of the nucleic acid containing position 908 of the ER gene. A suitable oligonucleotide primer pair for use in the first PCR is any oligonucleotide pair that generates a PCR product containing position 908 of the ER gene. Preferably, the oligonucleotide primer pair yields a PCR product of 50-5000 bp. In the examples described below, oligonucleotide primers (i.e., SEQ ID NO:5 and SEQ ID NO:6) were used that result in a 299 base pair (bp) fragment containing position 908. From this first PCR, both WT and mutant ER nucleic acids are amplified and present in the first population of PCR products.

The first population of PCR products is then subjected to a first restriction enzyme digest in which the restriction enzyme differentially digests the first and second nucleic acids based on a recognition sequence overlapping position 908. The restriction enzyme Mbo II is preferred for the first restriction digest, as it recognizes a restriction site overlapping position 908 in WT ER DNA (i.e., the second nucleic acid) but does not recognize a restriction site overlapping position 908 in mutant ER DNA (i.e., the first nucleic acid) due to the mutation. WT ER DNA, therefore, is digested at a site that mutant ER DNA is not. Digestion of WT ER DNA with such an enzyme reduces or eliminates WT DNA as template for PCR. Next, the first population of PCR products is subjected to a second PCR resulting in a second population of PCR products. As with the first PCR, any standard PCR conditions may be used that result in amplification of the nucleic acid containing position 908. A suitable oligonucleotide primer pair for use in the second PCR is any oligonucleotide primer pair that hybridizes to ER nucleic acid sequence internal to where the first oligonucleotide primer pair hybridizes to ER nucleic acid sequence, resulting in a second population of PCR products that are shorter than the first population of PCR products. In the examples described below, oligonucleotide primers (i.e., SEQ ID NO:7 and SEQ ID NO:8) were used that result in a 198 bp fragment containing position 908.

The second population of PCR products are then subjected to a second restriction enzyme digest in which the restriction enzyme differentially digests the first and second nucleic acids based on a recognition sequence overlapping position 908 and is different from the restriction enzyme in the first restriction enzyme digest. A preferred restriction enzyme for the second digest is Mnl I, as it recognizes a restriction site overlapping position 908 in the mutant ER DNA but does not recognize a restriction site overlapping position 908 in WT ER DNA. Therefore, when electrophoretically separated, products from the second PCR result in an electrophoretic pattern that distinguishes between mutant and WT ER DNA.

### Correlating Presence of a Mutant Nucleic Acid with Disease

The methods and compositions of the invention may be used to detect any disease or predisposition to a disease that can be correlated with a particular mutation. Such diseases include proliferative diseases of breast epithelial cells, including hyperplasia, atypia, carcinoma in situ, invasive breast cancer, and metastatic breast cancer. In the experiments below, a method for detecting a point mutation correlated with metastatic breast cancer is described. Variations of this method, however, may be used to detect any number of mutations that are correlated with a disease. To adopt the method described below to a particular mutation, oligonucleotide primers suitable for amplifying a nucleic acid containing that mutation and restriction enzymes that differentially digest this nucleic acid vs. a corresponding WT nucleic acid are selected. Oligonucleotide primer design, DNA sequence analysis, and restriction digest techniques are well known in the art.

### Kits

The invention also provides a kit for detecting a nucleic acid correlated with the presence of or predisposition to breast cancer in a human subject. An exemplary kit of the invention includes four oligonucleotide primers, two restriction enzymes and printed instructions for performing PCRs and restriction digests using the primers and enzymes of the kit. Two oligonucleotides (e.g., nucleotide sequences of SEQ ID NO:5 and SEQ ID NO:6) provided with the kit are used in a first PCR that amplifies nucleic acid sequence containing position 908 of the ER gene. Two additional oligonucleotides (e.g., nucleotide sequences of SEQ NO:7 and SEQ ID NO:8) provided with the kit are used in a second PCR that amplifies a nucleic acid sequence containing position 908 resulting in a population of PCR products that are shorter than the products of the first PCR. Any restriction enzymes that differentially cleave WT ER DNA versus ER DNA containing a mutation at position 908 may be used in a kit of the invention. Preferred restriction enzymes of the invention are Mbo II and Mnl I. Mbo II recognizes a restriction site at position 908 if the nucleotide at position 908 is WT (i.e., A). Mnl I recognizes a restriction site at position 908 if the nucleotide at position 908 is mutated to a G. In preferred versions of the kit, reagents for PCRs and restriction digests are included (e.g., buffer, polymerase, magnesium ions, deoxynucleotide triphosphates).

### EXAMPLES

The following examples serve to illustrate the invention without limiting it thereby. It will be understood that variations and modifications can be made without departing from the spirit and scope of the invention.

### Example 1

### ER DNA Assay for Cancer Diagnosis

### Methods

DNA containing the mutant (MUT) ER sequence is expected to be present in low concentrations in serum, and furthermore expected to be in low abundance relative to WT ER DNA. Given the predicted low abundance of the MUT ER DNA, methods such as allele-specific real-time quantitative PCR would not be expected to detect MUT ER DNA in a vast excess of WT ER DNA. Accordingly, a two-stage method based on standard nested PCR was designed that relies on differential restriction digestion of WT vs. MUT ER DNA using a WT-specific restriction endonuclease to reduce or eliminate the signal from WT ER DNA. Confirmation of MUT ER DNA is then completed by a separate distinct restriction enzyme digestion specific for only the MUT ER DNA.

### Sequence Information

The sequence of the ER in the region of interest is:

| | |
|---|---|
| WT— | |
| 5′-gcccgctcatgatcaaacgctctaaGAAGAaca | [SEQ ID NO: 1] |
| gccTggccttgtccctgacggccgaccag-3′ | |
| MUT— | |
| 5′-gcccgctcatgatcaaacgCtctaaGAGGaaca | [SEQ ID NO: 2] |
| gcctggccttgtccctgacggccgaccag-3′ | |

The position of the mutation at nucleotide 908 (A of initiator ATG=1) is underlined in the above sequence. The mutation is an A to G transition (i.e., A in WT, G in MUT). The restriction endonuclease recognition sites for cleaving WT and MUT ER DNA at position 908, respectively, are Mbo II: 5′-GAAGAnnnnn_n′-3′ [SEQ ID NO:3] and Mnl I: 5′-CCTCnnnnnn_n′3-[SEQ ID NO:4]. Referring to the Mbo II restriction site, for example, the underscore indicates that the bottom strand (which is not shown) is cleaved after the seventh nucleotide to the 3′ side of the recognition site. The apostrophe indicates that the top strand is cleaved 8 nucleotides to the 3′ side of the recognition site, leaving a 1-nucleotide 3′ overhang. The recognition site for Mbo II overlaps the position of the mutation. This site is present in WT ER, but lost in MUT ER. Conversely, the mutation creates a novel site for Mnl I not present in WT ER.

An example of a strategy for an ER DNA assay for cancer diagnosis of the invention involves the following steps. DNA is purified from serum, tissue, or from circulating tumor cells in blood. A first PCR reaction bracketing the mutation site is performed. The expected product is 299 bps. The resulting DNA includes both WT and MUT ER products. The first-round reaction products are digested with Mbo II to reduce or eliminate WT templates. A second PCR reaction internal to first primer pair (“nested” PCR) is performed. The expected product is 198 bps. The PCR product is digested with Mnl I. The presence of MUT ER DNA is indicated by restriction fragments of 146 and 54 bps. Residual WT ER DNA remains uncut by Mnl I.

Typical reaction conditions (25 uL Reactions) include the following components: 60 millimolar Tris pH 9.5, 15 millimolar ammonium sulfate, 7.5 millimolar MgCl₂, 10 picomoles Primer #1, 10 picomoles Primer #2, 200 micromolar dATP, 200 micromolar dCTP, 200 micromolar dTTP, 200 micromolar dGTP, 0.25 uL Gibco Platinum® Taq polymerase (Gibco, Carlsbad, Calif.), and DNA.

Standard cycling parameters are as follows: 95° C. for 2 minutes, 95° C. for 30 seconds /62° C. for 90 seconds/72° C. for 90 seconds ×30 cycles, 72° C. for 7 minutes, and 4° C. holding as necessary.

Examples of suitable primers are the following:

| | | |
|---|---|---|
| Outside (first round) primers: | | |
| 01: | | |
| 5′-agcgccagagagatgatggggaggg-3′ | [SEQ ID NO: 5] | (F) outside |
| 02: | | |
| 5′-aaagcctggcaccctcttcgcccag-3′ | [SEQ ID NO: 6] | (R) outside |
| Inside (second round) primers: | | |
| 03: | | |
| 5′-tgctggagacatgagagctgccaac-3′ | [SEQ ID NO: 7] | (F) inside |
| 04: | | |
| 5′-ttggtcagtaagcccatcatcgaag-3′ | [SEQ ID NO: 8] | (R) inside |
| Mutagenic Primer: | | |
| 5′-gccaggctgttcCtcttagagcgttt-3′ | [SEQ ID NO: 9] | (R) mutation creation |

1. Basic Test of PCR Primers.

The “outside” (first round) primers, the “inside” (second round) primers, and a mutagenic primer were all tested in a PCR under standard cycling parameters to demonstrate that they worked as expected. The “outside” primers were predicted to produce a 299 bp product, the “inside” primers were predicted to produce a 198 bp product, and the mutagenic primer (in combination with primer 01) was predicted to yield a 120 bp product. Each of these products was easily detected using 10 pg of input DNA. PCR reactions were performed using standard cycling parameters and reaction products were separated on a 2% agarose gel. Appropriately-sized products were easily detected with all primer pairs using 10 picograms of an ER-containing plasmid as input DNA.

2. Test of Nested PCR Strategy and Sensitivity.

DNA from this experiment was diluted and used in a “nested” PCR approach. The “outside” reaction products containing 1 picogram and 0.1 picogram of input DNA were diluted 1:10⁻¹, 1:10⁻², and 1:10⁻³ into standard PCR mix with “inside” primers, and amplified 30 cycles. The expected 198 bp product was detected at all dilutions of the 1 picogram reaction. PCR products from the basic test of PCR primers experiment were diluted as indicated and re-amplified using “inside” (nested) primers. All dilutions of the 1 picogram reaction yielded a 198 bp product as expected.

3. Creation of MUT Template Using a Megaprimer Strategy.

A classic “megaprimer” approach was used to generate a 299 bp PCR product carrying the A →4 mutation for use as a positive control. The 120 bp product from the reaction containing the mutagenic primer from the basic test of PCR primers experiment was used as a “megaprimer” in combination with primer 02 to create a 299 bp product that now carries the mutation. ER DNA (100 ng) was amplified using 2 uL of the 120 bp PCR product from the basic test of PCR primers experiment (100 and 10 picogram reactions) in combination with primer 02 for 30 cycles. This reaction was diluted to 0.1 and 0.01 and reamplified with primers 01 and 02 to increase the yield of mutant product. This reaction yielded the expected 300 bp product.

To demonstrate that this PCR product was indeed carrying the mutation, the PCR product was digested with Mnl I and Mbo II. This DNA was found to have the expected digestion pattern for the mutation-specific enzyme Mnl I. WT DNA gave a different pattern. The amplified DNA products from the megaprimer reaction and control WT reaction were digested with Mnl I or Mbo II to confirm the presence of the newly-introduced mutation. The expected 83 bp restriction fragment was observed.

Additional development of this protocol prior to use in a clinical laboratory setting may include testing patient serum samples, tissue samples, and circulating epithelial cell samples.

The mutant estrogen receptor nested PCR-restriction enzyme DNA assay of the present invention provides a novel, unique, specific, and sensitive assay for reliably detecting the ER mutation. Unlike the published methodology using direct genomic sequence analysis that is laborious and time-consuming, this mutant ER nested PCR-restriction enzyme DNA assay is much more amenable to the high-throughput analysis that is essential for clinical application. The use of mutation-specific restriction enzymes ensures the necessary specificity.

### Example 2

### Detection of Mutant ER DNA in Plasma of Metastatic Breast Cancer

### Patients

Using the mutant ER DNA assay described above, DNA purified from plasma samples from metastatic breast cancer patients enrolled in a trial of second-line hormone therapy was screened. From 39 patient plasma samples, 7 were positive for the mutant estrogen receptor (18%).

### Example 3

### Determining Sensitivity of the Assay

The sensitivity of the assay was determined. Using a single round of PCR with the first-round (“outside”) primers, 0.35 fg (350 attograms; approximately 60 molecules of ER DNA) was routinely detected. Occasionally, 35 attograms was detected. ER DNA was subjected to a single round of PCR using the first-round primers (which yield a 300 bp product, as expected). Input DNA amounts from 500 to 1 fg were used to generate PCR products. PCR products from 50 to 0.0035 fg (3.5 attograms) were observed. The expected product was easily detected down to 350 attograms, and detectable in some reactions at 35 attograms. Reactions in the 0.35 to 0.0035 fg range were run in duplicate in this experiment.

The ability to detect mutant DNA in a background of wt DNA under sensitive conditions was examined. It was shown that 20 fg of mutant DNA can be easily detected in a background of 200 fg of wt DNA using standard ethidium bromide staining. WT and mutant ER DNAs were mixed and subjected to the standard two-round nested PCR described above. The products were then digested with Mnl I, which specifically digests the mutant DNA, yielding fragments of 145 bp and 53 bp; wt DNA is not digested by this enzyme, yielding a fragment of 198 bp. Digestion products were separated by agarose gel electrophoresis. Bands corresponding to mutant DNA were readily visible down to 20 fg of mutant in the presence of 200 fg of wt DNA, and a faint product was detectable at 2 fg of mutant DNA. Hybridization techniques would, of course, extend this sensitivity further.

### Example 4

### Additional Oligonucleotides for a Mutant ER Nested PCR-Restriction

### Enzyme DNA Assay

The following primers are used in the same fashion as the oligonucleotide primers described above (i.e., SEQ ID NOs:5-8):

| | | |
|---|---|---|
| 5′-gcatccaacaaggcactgaccatct-3′ | [SEQ ID NO: 10] | Leit06 (a “reverse” primer) |
| 5′-actcggaatagagtatggggggctc-3′ | [SEQ ID NO: 11] | Leit07-(a “reverse” primer) |
| 5′-tggccaagcccgctcatgatcaaac-3′ | [SEQ ID NO: 12] | Leit08-(a “forward” primer) |

These primers are used in the following pairs: Primers Leit03 (SEQ ID NO:7) and Leit07 (SEQ ID NO:11) replace Leit01 (SEQ ID NO:5) and Leit02 (SEQ ID NO:6), (respectively). Leit03/Leit07 (SEQ ID NO:7/SEQ ID NO:11) are used in the first round PCR, generating a 146 bp product. Primers Leit08 (SEQ ID NO:12) and Leit06 (SEQ ID NO:10) replace Leit03 (SEQ ID NO:7) and Leit04 (SEQ ID NO:8), (respectively). Leit08/Leit06 (SEQ ID NO:12/SEQ ID NO:10) are used in the second round PCR, generating a 92 bp product.

The use of these primers would have two advantages over the previously described primers: a) DNA in serum is known to be degraded, with fragment sizes typically averaging 200 bps or less. These new primers were chosen to anneal closer to the site of the mutation, allowing DNA that is shorter than 200 bps to be efficiently amplified, and thus increasing the sensitivity of the assay, particularly in samples with more than average degradation; and b) certain single nucleotide polymorphism (SNP) detector kits, which, in combination with the technology described herein, could form the basis of an integrated clinical amplification and detection system (e.g., Mutector™ kit by Trimgen, Sparks, Md.) require precise spacing between the second round reverse primer and the mutation. Primer Leit 06 (SEQ ID NO:10) was designed with this downstream application in mind.

In summary, an assay was developed to screen plasma and serum samples for mutant estrogen receptor DNA. This assay may be used to screen large numbers of patient samples to determine if the presence of mutant estrogen receptor DNA in the serum or plasma correlates with clinical parameters.

### Other Embodiments

While the above specification contains many specifics, these should not be construed as limitations on the scope of the invention, but rather as examples of preferred embodiments thereof. Many other variations are possible. Accordingly, the scope of the invention should be determined not by the embodiments illustrated, but by the appended claims and their legal equivalents.

What is claimed is:

1. A method for detecting a nucleic acid correlated with the presence of or predisposition to acquiring breast cancer in a human subject, the method comprising the step of:
detecting in a bodily fluid of the subject a nucleic acid comprising an A to G transition at nucleotide 908 in the estrogen receptor gene, wherein the presence of the nucleic acid comprising an A to G transition at nucleotide 908 indicates the subject has or is predisposed to acquiring breast cancer.

2. The method of claim 1, wherein the bodily fluid comprises blood or a blood fraction.

3. The method of claim 1, wherein the bodily fluid comprises plasma.

4. The method of claim 1, wherein the bodily fluid comprises serum.

5. The method of claim 1, wherein less than 10 femtograms of the nucleic acid comprising an A to G transition at nucleotide 908 in the ER gene is present in the bodily fluid.

6. The method of claim 1, wherein less than 1 femtogram of the nucleic acid comprising an A to G transition at nucleotide 908 in the ER gene is present in the bodily fluid.

7. The method of claim 1, wherein less than 0.1 femtogram of the nucleic acid comprising an A to G transition at nucleotide 908 in the ER gene is present in the bodily fluid.

8. The method of claim 1, wherein the method of detecting the nucleic acid comprises;
(a) subjecting nucleic acids from the bodily fluid to a first polymerase chain reaction resulting in a first population of polymerase chain reaction products comprising at least a first nucleic acid having an A to G transition at nucleotide 908 in the estrogen receptor gene and a second nucleic acid WT at nucleotide 908 in the estrogen receptor gene;
(b) subjecting the first population of polymerase chain reaction products to a first restriction enzyme digest, wherein the enzyme recognizes a restriction site overlapping nucleotide 908 in the second nucleic acid but not in the first nucleic acid;
(c) subjecting the first population of polymerase chain reaction products to a second polymerase chain reaction resulting in a second population of polymerase chain reaction products; and
(d) subjecting the second population of polymerase chain reaction products to a second restriction enzyme digest, wherein the restriction enzyme recognizes a restriction site overlapping nucleotide 908 in the first nucleic acid but not in the second nucleic acid and is different from the restriction enzyme in the first restriction enzyme digest.

9. The method of claim 1, wherein the first polymerase chain reaction comprises a first oligonucleotide primer and a second oligonucleotide primer, the first oligonucleotide primer comprising the nucleotide sequence of SEQ ID NO:5, and the second oligonucleotide primer comprising the nucleotide sequence of SEQ ID NO:6.

10. The method of claim 1, wherein the second polymerase chain reaction comprises a third oligonucleotide primer and a fourth oligonucleotide primer, the third oligonucleotide primer comprising the nucleotide sequence of SEQ ID NO:7, and the fourth oligonucleotide primer comprising the nucleotide sequence of SEQ ID NO:8.

11. The method of claim 1, wherein nucleic acid fragments of the second restriction enzyme digest are electrophoretically separated to yield an electrophoretic pattern specific to the digestion of the first nucleic acid.

12. The method of claim 11, wherein the electrophoretic pattern specific to the first nucleic acid indicates the presence of the first nucleic acid in the bodily fluid.

13. The method of claim 12, wherein the presence of the first nucleic acid in the bodily fluid indicates that the bodily fluid contains a breast cancer.

14. A kit for detecting a nucleic acid correlated with the presence of breast cancer, or a precancerous condition of the breast, or a predisposition to acquire breast cancer in a human subject, the kit comprising:
(a) a first pair of oligonucleotides for amplifying a portion of the estrogen receptor gene;
(b) a second pair of oligonucleotides for amplifying a nucleic acid within the portion of the estrogen receptor gene amplified by the first pair of oligonucleotides;
(c) a first restriction enzyme;
(d) a second restriction enzyme; and
(e) printed instructions for performing polymerase chain reactions using the first and second pairs of oligonucleotides and restriction digests using the first and second restriction enzymes.
